(19) United States
(12) Patent Application Publication
(10) Pub. No.: US 2026/0311846 A1
(43) Pub. Date: Oct. 8, 2026

(54) INFORMATION PROCESSING METHOD, INFORMATION PROCESSING DEVICE, AND INFORMATION PROCESSING SYSTEM

(71) Applicant: Casio Computer Co., Ltd., Tokyo (JP)

(72) Inventor: Nobuhiro AOKI, Tokyo (JP)

(73) Assignee: Casio Computer Co., Ltd., Tokyo (JP)

(21) Appl. No.: 19/634,879

(22) Filed: Mar. 31, 2026

(30) Foreign Application Priority Data
Apr. 2, 2025 (JP) 2025-061470

(51) Int. Cl. G16H 30/20; G06F 3/04842; G06F 21/62; G06F 3/12

(52) U.S. Cl. CPC G16H 30/20; G06F 3/04842; G06F 21/6254; G06F 3/1222; G06F 3/1238

(57) ABSTRACT

An information processing method involves receiving, from a user, a selection to anonymize or not to anonymize file information, and outputting the file information without anonymization in response to the selection not to anonymize the file information is received, or outputting the anonymized file information in response to the selection to anonymize the file information is received.

### CROSS REFERENCE TO RELATED APPLICATION

This application claims the benefit of Japanese Patent Application No. 2025-061470, filed on Apr.2, 2025, the entire disclosure of which is incorporated by reference herein.

### FIELD OF THE INVENTION

This application relates to an information processing method, an information processing device, and an information processing system.

### BACKGROUND OF THE INVENTION

Techniques for managing medical diagnostic information have been known. For example, Unexamined Japanese Patent Application Publication No. 2016-224793 discloses a medical information management apparatus that enables a case search with high accuracy using image comparison.

### SUMMARY OF THE INVENTION

An information processing method according to an aspect of the present disclosure involves: receiving, from a user, a selection to anonymize or not to anonymize file information; and outputting the file information without anonymization in response to the selection not to anonymize the file information is received; or outputting the anonymized file information in response to the selection to anonymize the file information is received.

### BRIEF DESCRIPTION OF DRAWINGS

A more complete understanding of this application can be obtained when the following detailed description is considered in conjunction with the following drawings, in which:

FIG. 1 is a block diagram illustrating a configuration of an information output device according to an embodiment;

FIG. 2 illustrates an example of an image data piece according to the embodiment;

FIG. 3 illustrates an example of a viewer screen according to the embodiment;

FIG. 4 illustrates an example of a diagnostic record screen according to the embodiment;

FIG. 5 illustrates an example of an output setting screen according to the embodiment;

FIG. 6 illustrates an example including non-anonymized data files and data folders output from the information output device according to the embodiment;

FIG. 7 illustrates an example of a non-anonymized CSV file output from the information output device according to the embodiment;

FIG. 8 illustrates an example including anonymized data files and data folders output from the information output device according to the embodiment;

FIG. 9 illustrates an example of an anonymized CSV file output from the information output device according to the embodiment;

FIG. 10 is a flowchart illustrating a file output process executed by the information output device according to the embodiment;

FIG. 11 illustrates an example of an image comparison screen according to the embodiment;

FIG. 12 illustrates an example of a print setting screen according to the embodiment;

FIG. 13 illustrates a first example of a print image output from the information output device according to the embodiment;

FIG. 14 illustrates a second example of a print image output from the information output device according to the embodiment; and

FIG. 15 is a flowchart illustrating a print output process executed by the information output device according to the embodiment.

### DETAILED DESCRIPTION OF THE INVENTION

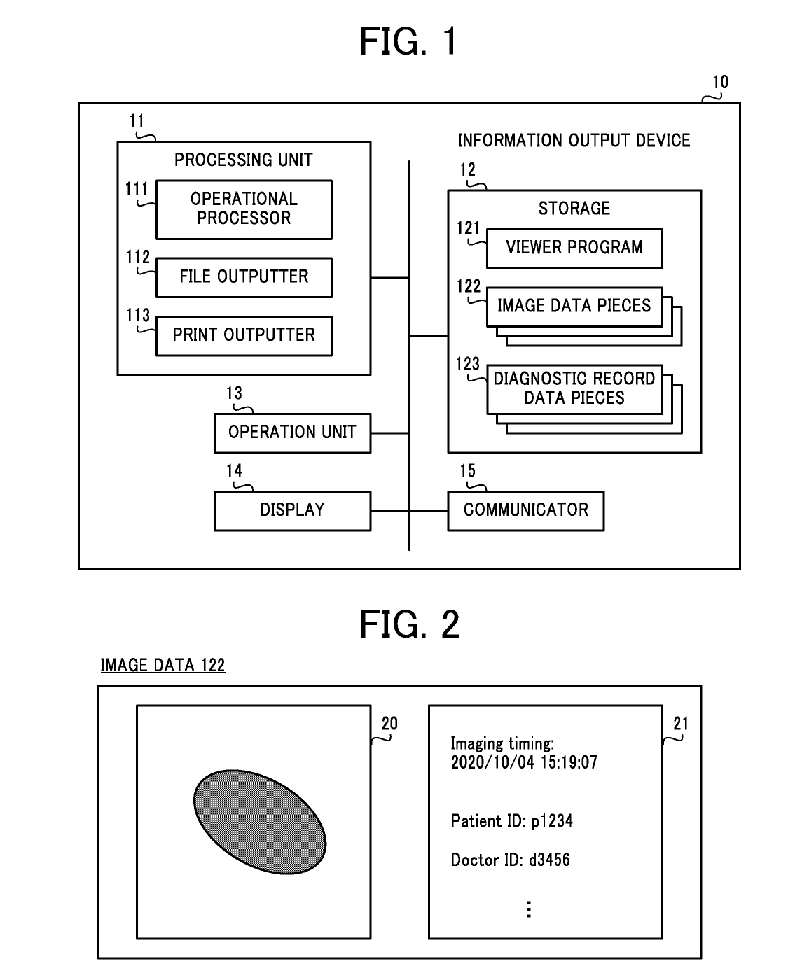

The following describes an embodiment of the present disclosure with reference to the accompanying drawings. In the drawings, the components identical or corresponding to each other are provided with the same reference symbol. An information output device 10 according to the embodiment is a medical support device that outputs medical diagnostic information to support diagnosis performed by a user, such as doctor or healthcare worker. In particular, the information output device 10 according to the embodiment organizes and adjusts diagnostic information on subjects and outputs the resulting information as output information, to help the user to view and manage the diagnostic information on the subjects, for example. Specific examples of the information output device 10 include information processing devices, such as personal computers and tablets, operated by the user. As illustrated in FIG. 1, the information output device 10 includes a processing unit 11, a storage 12, an operation unit 13, a display 14, and a communicator 15.

The processing unit 11 includes a central processing unit (CPU), read only memory (ROM), and random access memory (RAM). The CPU is a central calculation unit including a microprocessor and other components to execute various processes and calculations. In the processing unit 11, the CPU reads a control program stored in the ROM, and controls the overall operation of the information output device 10 by using the RAM as working memory. The processing of the processing unit 11 may be enabled by a single CPU alone or by multiple CPUs. The processing unit 11 may include an image processor, such as digital signal processor (DSP) or graphics processing unit (GPU).

The storage 12 is non-volatile memory, such as flash memory or hard disk. The storage 12 stores programs and data to be executed by the processing unit 11, and data generated by the processing unit 11. Specifically, the storage 12 stores a viewer program 121, image data pieces 122, and diagnostic record data pieces 123. The details of the stored program and data are described below. The operation unit 13 includes an input device, such as keyboard, mouse, or touch panel, and receives operations input from the user. The display 14 includes a liquid crystal or organic electroluminescence (EL) display, for example, and displays various images under the control of the processing unit 11. The communicator 15 includes a communication interface to communicate with external devices of the information output device 10. For example, the communicator 15 communicates with external devices, such as camera, printer, and cloud server, pursuant to a communication standard of the Internet, local area network (LAN), or universal serial bus (USB), for example.

The processing unit 11 includes, as functional components, an operational processor 111, a file outputter 112, and a print outputter 113. The CPU serves as these functional components of the processing unit 11 by loading a program stored in the ROM into the RAM and executing the program to control the operation. The functional components of the processing unit 11 may be enabled by a single CPU, or multiple CPUs cooperating with each other. The operational processor 111 receives an operation of the user via the operation unit 13 and executes a process in accordance with the received operation. The user indicates a diagnostician, such as doctor or healthcare worker, who diagnoses a subject. The subject indicates a person (diagnostic target), such as patient or examinee, to be diagnosed. Specifically, the operational processor 111 executes the viewer program 121 in accordance with an operation of the user. The viewer program 121 is a computer program or software for managing images and thus helping the user to view and manage the image data pieces 122 stored in the storage 12. The viewer program 121 is preinstalled in the information output device 10 and stored in the storage 12.

Each of the image data pieces 122 indicates a captured image to be diagnosed. For example, each image data piece 122 contains a captured image 20 and image-associated information 21, as illustrated in FIG. 2. The captured image 20 is an image that captures at least a part of the subject’s body. Specifically, the captured image 20 is a diagnostic image that captures a candidate lesion existing on the subject’s skin to facilitate diagnosis of the candidate lesion. The candidate lesion indicates a portion of the subject’s body that may include a pathological change, in other words, a disease, such as tumor. Examples of the candidate lesion include unusual features on the skin, such as moles, bruises, and flecks.

The captured images 20 in the image data pieces 122 are images that capture candidate lesions existing in various subjects’ bodies. For example, some of the captured images 20 may be images of the identical candidate lesion existing in the identical subject’s body captured at different timings or in different imaging conditions. For another example, others of the captured images 20 may be images of candidate lesions existing in different subjects’ bodies. That is, the image data pieces 122 indicate the captured images 20 each capturing any of different subjects.

The image-associated information 21 is related to each captured image 20. In more detail, the image-associated information 21 is information (metadata) that is generated when the camera captures the image 20 and accompanies the captured image 20. For example, the image-associated information 21 corresponds to exchangeable image file format (EXIF: registered trademark) information. Specifically, as illustrated in FIG. 2, the image-associated information 21 contains an imaging timing indicating the date and time of image capturing by the camera, a patient ID, which is an example of identification information on the subject captured in the captured image 20, and a doctor ID, which is an example of identification information on the diagnostician who captures the image 20. The image-associated information 21 may also contain an imaging location, camera settings, and other information, which are not illustrated. The camera settings include the diaphragm and shutter speed of the camera. The images 20 are captured by a medical camera, such as dermoscopic camera. Specifically, the diagnostician, such as doctor, captures an image of a candidate lesion on the body of the subject, such as patient, with the camera in a diagnostic field. In the image capturing, the diagnostician inputs the subject’s patient ID and the own doctor ID to the camera. The input patient and doctor IDs, imaging timing, and other information are thus recorded in the image-associated information 21 accompanying the captured image 20. The operational processor 111 communicates with the camera via the communicator 15 to acquire the image data pieces 122 containing the images 20 captured by the camera and the corresponding image-associated information 21, and then stores the acquired image data pieces 122 in the storage 12.

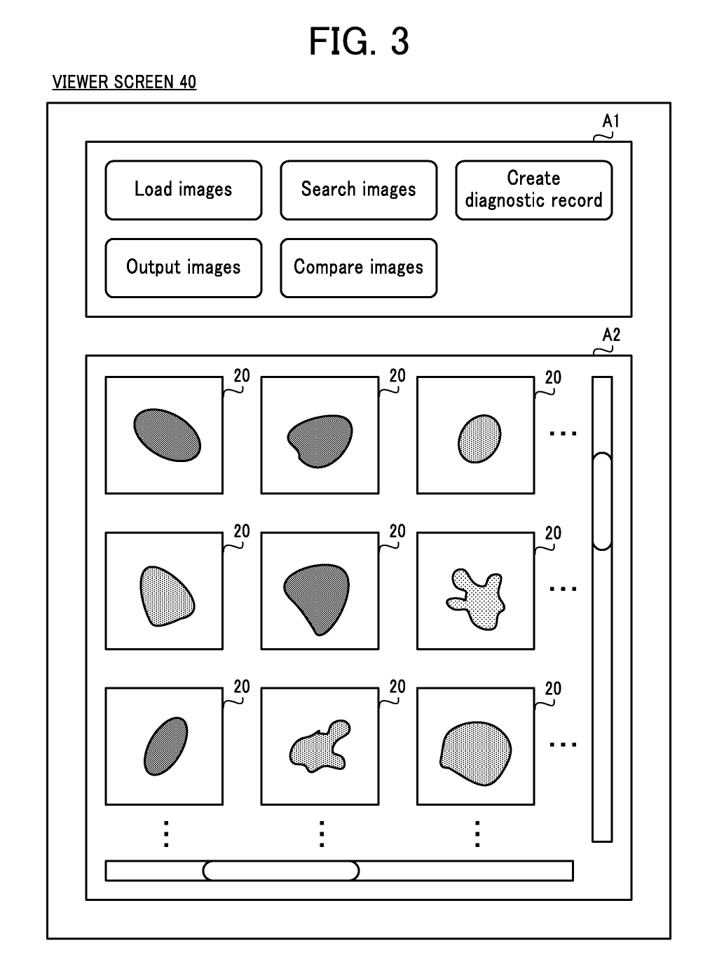

The user, who wants to view or manage such image data pieces 122, operates the operation unit 13 to activate the viewer program 121 stored in the storage 12. After activation of the viewer program 121, the operational processor 111 causes the display 14 to display a viewer screen 40 illustrated in FIG. 3. The viewer screen 40 helps the user to view and manage the image data pieces 122 stored in the storage 12. Specifically, the viewer screen 40 includes an operation field A1 to receive an operation of the user, and an image field A2 to show the captured images 20.

The operation field A1 includes items “Load images”, “Search images”, “Create diagnostic record”, “Output images”, “Compare images” and other items selectable by the user. The item “Load images” is selected to load the image data pieces 122 stored in the storage 12 onto the viewer screen 40. When the user selects the item “Load images” by operating the operation unit 13, the operational processor 111 loads the image data pieces 122 stored in the storage 12 onto the viewer screen 40, and causes the captured images 20 contained in the loaded image data pieces 122 to appear in the image field A2.

The item “Search images” is selected to retrieve a target captured image 20 from the captured images 20 indicated by the image data pieces 122 loaded on the viewer screen 40. When the user selects the item “Search images” by operating the operation unit 13, the user is allowed to input a retrieval keyword, such as imaging timing, patient ID, or doctor ID. The operational processor 111 searches for a captured image 20 associated with the retrieval keyword input by the user, with reference to the imaging timings, patient IDs, doctor IDs, and other information contained in the image-associated information 21. When the searching process successfully retrieves at least one captured image 20 associated with the retrieval keyword, the operational processor 111 causes the retrieved image 20 to appear in the image field A2.

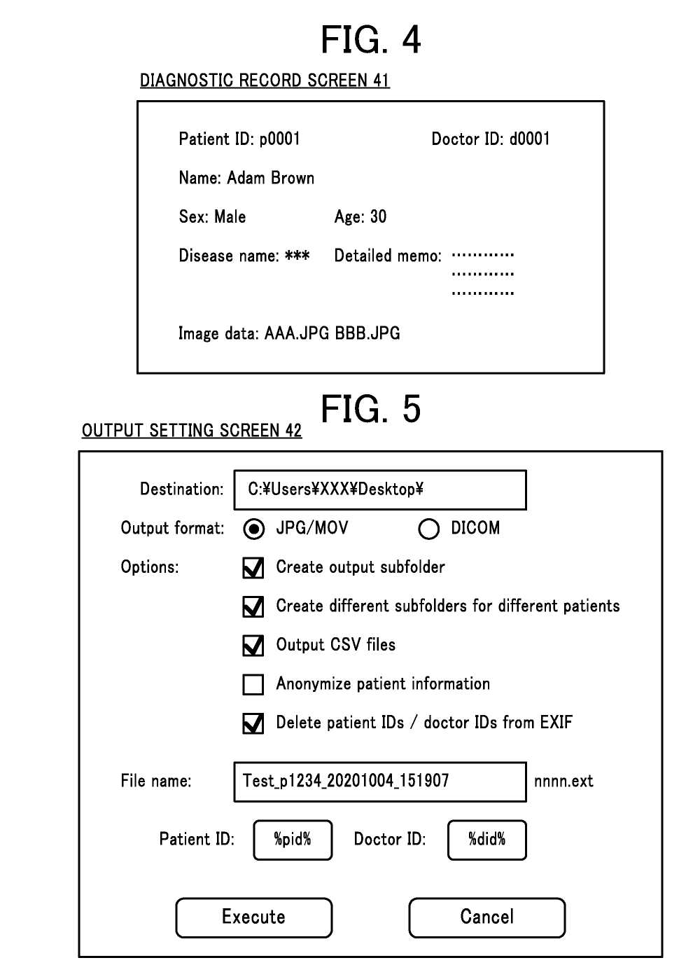

The item “Create diagnostic record” is selected to create a record regarding diagnosis of the subject. When the user selects the item “Create diagnostic record” by operating the operation unit 13, the operational processor 111 causes the display 14 to display a diagnostic record screen 41 illustrated in FIG. 4, for example. The diagnostic record screen 41 allows the user to input various diagnostic data on each subject. For example, the diagnostic record screen 41 allows the user to input the patient ID, name, sex, age, and other information on the subject of interest, the doctor ID of the diagnostician who diagnoses the subject, a diagnosed disease name, and a detailed memo, as illustrated in FIG. 4. The diagnostic record screen 41 also allows the user to input data file names of the image data pieces 122 and other information to associate the image data pieces 122 indicating the captured images 20 of the same subject with one another. The operational processor 111 receives the data input by the user on the diagnostic record screen 41, generates diagnostic record data pieces 123 for the individual subjects, and stores the generated diagnostic record data pieces 123 in the storage 12. The diagnostic record data pieces 123 serve as history data indicating the past diagnostic results like electronic charts.

Referring back to FIG. 3, the items “Output images” and “Compare images” on the viewer screen 40 are selected to output medical diagnostic information. The processing unit 11 of the information output device 10 can execute a first output process corresponding to the item “Output images” and a second output process corresponding to the item “Compare images”, as processes of organizing or adjusting diagnostic information on the subjects and outputting the resulting information. The item “Output images” on the viewer screen 40 is selected to output the captured images 20 as data files. The user selects one or more captured images 20 to be output, by operating the operation unit 13, from among the captured images 20 appearing in the image field A2. Then, when the user selects the item “Output images”, the file outputter 112 executes the first output process. In the first output process, the file outputter 112 outputs image files or data files indicating the captured images 20 of the subjects, as output information. The image files are examples of file information (output information as data files) output from the information output device 10.

In more detail, the information output device 10 retains the image data pieces 122 and the diagnostic record data pieces 123 in the storage 12, as diagnostic information on the subjects. These information pieces are difficult to manage when stored in different data formats or in various locations. In particular, as the information pieces related to more subjects or more diagnostic processes increase in number and size, the information pieces make it difficult for the user to view a pinpoint piece of information at the necessary timing. To avoid this problem, the file outputter 112 converts the image data pieces 122 and the diagnostic record data pieces 123 stored in the storage 12 into data files having file names and formats easily manageable by the user, and outputs the resulting data files to data folders easily manageable by the user.

Specifically, when the user selects the item “Output images” on the viewer screen 40, the file outputter 112 causes the display 14 to display an output setting screen 42 illustrated in FIG. 5. The output setting screen 42 allows the user to define settings for outputting one or more image files indicating the captured images 20 selected to be output. The output setting screen 42 allows the user to set and input a destination data folder to which each image file is output, an output format of the image file, and a file name of the image file. The items “Patient ID” and “Doctor ID” on the output setting screen 42 represent input buttons to be selected to add variables depending on the captured images 20 to the file names. While the user can freely input the file name of each image file, the user can also easily add the subject’s patient ID or the diagnostician’s doctor ID related to the captured image 20 to the file name by selecting the item “Patient ID” or “Doctor ID”. Information on these variables is available from the image-associated information 21 accompanying the captured image 20 selected to be output, or the diagnostic record data piece 123.

In accordance with the settings defined by the user on the output setting screen 42, the file outputter 112 outputs the image file indicating the captured image 20 selected to be output. Specifically, the file outputter 112 reads the image data piece 122 on the captured image 20 selected to be output, from the image data pieces 122 stored in the storage 12. The file outputter 112 then converts the read image data piece 122 into an image file having the set output format and file name, and outputs the converted file to the set destination data folder. The output image file is a data file containing the selected captured image 20, and the image-associated information 21 accompanying the captured image 20 in the image data piece 122.

In more detail, when a single captured image 20 is selected to be output, the file outputter 112 outputs a single image file indicating the selected captured image 20. In contrast, when multiple captured images 20 are selected to be output, the file outputter 112 outputs a number of image files each indicating one of the selected captured images 20, corresponding to the number of selected captured images 20. In such a case where multiple image files are output, the file outputter 112 may automatically assign mutually different numbers (for example,”nnnn” contained in the file name in FIG. 5) to the individual file names to ensure that all the image files have unique file names. Alternatively, the file outputter 112 may add patient or doctor IDs, imaging timings, or other variables to the file names, as described above, to ensure the uniqueness of the file names among the image files.

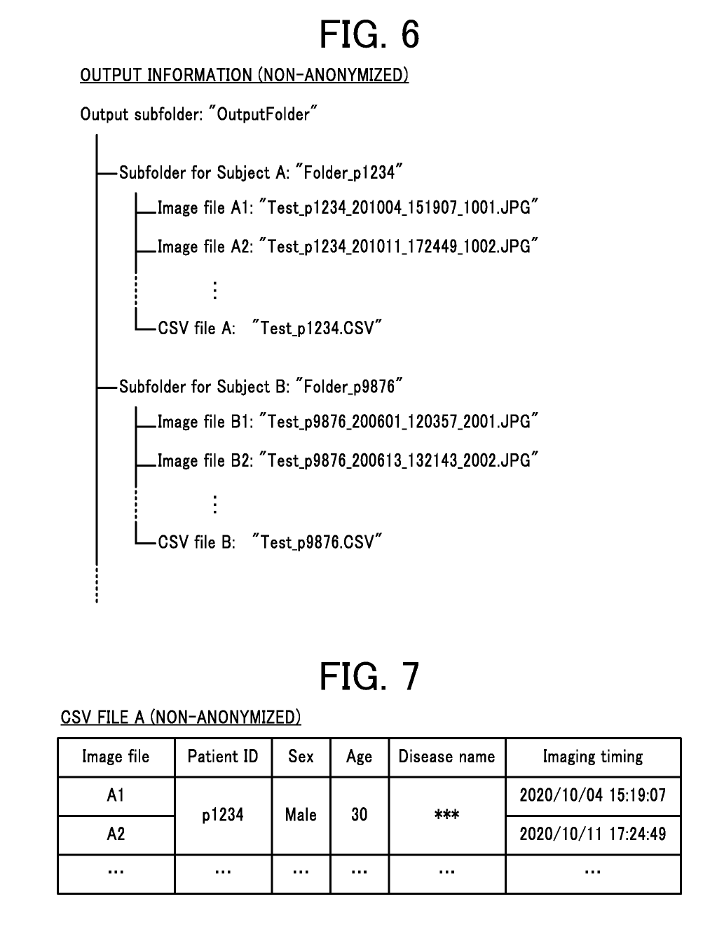

In addition, the file outputter 112 receives output options set by the user on the output setting screen 42. The output options are items that allow the user to select whether to apply the items to the output modes of image files. In the example in FIG. 5, the user can select five options by checking their checkboxes. When the user selects some of the options and selects the item “Execute”, the file outputter 112 outputs an image file indicating the captured image 20 selected to be output, in accordance with the options selected by the user. Each of the options is described below with reference to FIGS. 6 to 9. FIG. 6 illustrates configurations of data files and data folders, as the output information from the file outputter 112, when the file outputter 112 does not execute anonymization, which is described below. Specifically, FIG. 6 illustrates an example in which the captured images 20 of subjects A, B, ... are selected to be output, and the file outputter 112 outputs image files A1, A2, ... indicating captured images 20 of the subject A and image files B1, B2, ... indicating captured images 20 of the subject B.

The first option “Create output subfolder” on the output setting screen 42 allows the user to select whether to create a new data subfolder to which image files are output, in the destination data folder set on the output setting screen 42. When the first option is selected, the file outputter 112 creates a data subfolder having an appropriate folder name (“OutputFolder” in the example in FIG. 6) in the destination data folder set on the output setting screen 42. In contrast, when the first option is not selected, the file outputter 112 does not create such a data subfolder.

The second option “Create different subfolders for different patients” on the output setting screen 42 allows the user to select, when captured images 20 of multiple subjects are selected to be output, whether to sort the image files into data subfolders for the individual subjects. When the second option is selected, the file outputter 112 creates data subfolders for the individual subjects, like the subfolder for the subject A, the subfolder for the subject B, ... illustrated in FIG. 6. In other words, the file outputter 112 creates data subfolders for the subjects A, B, ... on a one-to-one basis. The file outputter 112 then outputs each image file to the data subfolder associated with the subject of the image file, among the created data subfolders. Specifically, the file outputter 112 outputs the image files A1, A2, ... indicating the captured images 20 of the subject A to the subfolder for the subject A, and outputs the image files B1, B2, ... indicating the captured images 20 of the subject B to the subfolder for the subject B. That is, when the second option is selected, the file outputter 112 creates data subfolders for the individual subjects, and sorts and outputs the image files into the data subfolders. The image files for multiple subjects are thus sorted into data subfolders for the individual subjects and can be easily managed by the user. In contrast, when the second option is not selected, the file outputter 112 outputs all the image files A1, A2, ... , B1, B2, ... to a single output subfolder without creating data subfolders for the individual subjects.

The third option “Output CSV files” on the output setting screen 42 allows the user to select whether to output comma-separated values (CSV) files as output data files in addition to the image files. The CSV files are data files that indicate diagnostic information on the subjects in the form of text (character strings). Specifically, the CSV files are generated by organizing the diagnostic information on the subjects recorded in the diagnostic record data pieces 123, in the CSV format easily manageable by the user. When the third option is selected, the file outputter 112 outputs, as the output information, CSV files indicating diagnostic information on the subjects as well as the image files indicating captured images of the subjects. Specifically, as illustrated in FIG. 6, the file outputter 112 outputs a CSV file A indicating diagnostic information on the subject A as well as the image files A1, A2, ... , and a CSV file B indicating diagnostic information on the subject B as well as the image files B1, B2, ... . The CSV files are examples of the file information (output information as data files) output from the information output device 10.

For example, FIG. 7 illustrates the CSV file A for the subject A in a table form. The CSV file A for the subject A contains the patient ID, sex, age, disease name, and other information on the subject A, as illustrated in FIG. 7. The CSV file A also contains, for the individual image files A1, A2, ... related to the subject A, the imaging timings and other information on the captured images 20 of the subject A. The CSV file B for the subject B also contains both information on the subject B and information on the captured images 20 of the subject B, although the configuration of the CSV file B is not illustrated. The file outputter 112 creates a CSV file having such content for each subject, in accordance with the information recorded in the diagnostic record data piece 123 on the subject. The file outputter 112 outputs the created CSV file to the same data folder to which the image files for the subject are output.

In contrast, when the third option is not selected, the file outputter 112 does not output such CSV files. Alternatively, the file outputter 112 may output CSV files, not for the individual subjects, but for the individual image files. In other words, the file outputter 112 may output a separate CSV file for each of the image files A1, A2, ... , B1, B2, ... , instead of a CSV file for each subject. The user may be allowed to select whether to output CSV files for the individual subjects or for the individual image files, as one of the options on the output setting screen 42.

Referring back to FIG. 5, the fourth option “Anonymize patient information” on the output setting screen 42 allows the user to select whether to anonymize output information. Anonymization of output information means converting or deleting specific information pieces in the image files and CSV files, corresponding to the output information from the file outputter 112, to make the subjects unidentifiable from the output information. Specifically, the output information from the file outputter 112 contains the captured images 20, the disease name, and other sensitive information on the subjects. In some cases, the output information needs to be anonymized for personal information protection. In other cases, the output information for some uses needs to contain personal information on the subjects without anonymization. Exemplary situations requiring anonymization include using images of candidate lesions in a scientific paper and printing only images of candidate lesions. In contrast, exemplary situations not requiring anonymization include introducing the output information in electronic charts, using the output information for consultation with another doctor, and using the output information in statistics research. In view of these backgrounds, the file outputter 112 of the information output device 10 according to the embodiment receives a user’s selection of the fourth option regarding whether to anonymize the output information. The fourth option allows the user to flexibly select whether to anonymize the output information, depending on the use of the output information.

When receiving a selection not to anonymize the output information through the fourth option, the file outputter 112 does not execute anonymization and outputs non-anonymized output information. First, when no anonymization is to be executed, the file outputter 112 outputs image files having file names containing identification information on the subjects. The identification information on the subject indicates information from which the subject is identifiable, in other words, information for identifying who the subject is. Specific examples of the identification information include the patient ID and the name of the subject. Examples of the identification information in this embodiment do not include the subject’s age, sex, or disease name, because another user cannot easily identify the subject directly from these information pieces alone.

Specifically, when no anonymization is to be executed, the file outputter 112 does not delete the patient IDs from the file names, defined by the user on the output setting screen 42, of the image files to be output. In the example in FIG. 6, the file names of the image files A1, A2, ... contain a patient ID “p1234” of the subject A, and the file names of the image files B1, B2, ... contain a patient ID “p9876” of the subject B. When no anonymization is to be executed, the file outputter 112 maintains the file names containing such patient IDs without any change. In the case where the file names defined by the user contain no patient IDs, the file outputter 112 may maintain the file names, or add corresponding patient IDs of the subjects to the set file names such that each file name contains the patient ID.

Second, when the above-described second option is selected in the case of no anonymization, the file outputter 112 creates data subfolders for the individual subjects having folder names each containing the patient ID of the corresponding one of the subjects. Specifically, as illustrated in FIG. 6, the file outputter 112 creates, as the data subfolder for the subject A, a data subfolder having the folder name “Folder_p1234” containing the patient ID of the subject A. The file outputter 112 also creates, as the data subfolder for the subject B, a data subfolder having the folder name “Folder_p9876” containing the patient ID of the subject B.

Third, when the above-described third option is selected in the case of no anonymization, the file outputter 112 creates CSV files for the individual subjects that contain the patient IDs of the corresponding subjects. Specifically, as illustrated in FIG. 7, the file outputter 112 creates, as the CSV file A for the subject A, a CSV file containing the patient ID “p1234” of the subject A as one information item. The file outputter 112 also creates, as the CSV file B for the subject B, a CSV file containing the patient ID “p9876” of the subject B as one information item, although the configuration of the CSV file B is not illustrated. The file outputter 112 adds the patient ID of each subject to the file name (“Test_p1234.CSV”, “Test_p9876.CSV”, ... in the example in FIG. 6) of the CSV file for this subject.

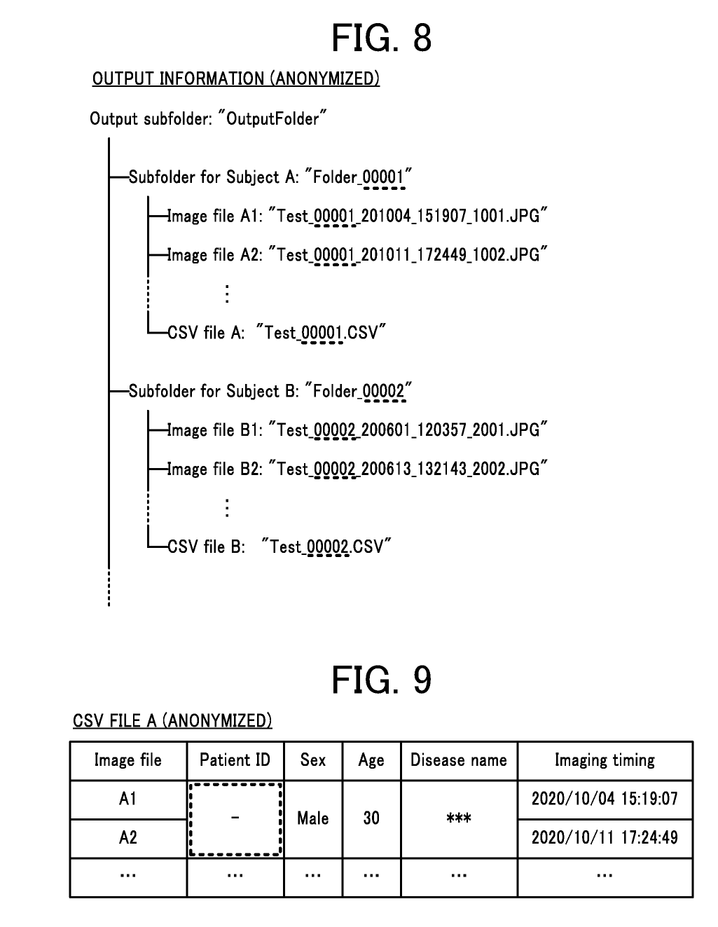

In contrast, when receiving a selection of the fourth option to anonymize the output information, the file outputter 112 executes anonymization and outputs the anonymized output information. FIG. 8 illustrates configurations of anonymized data files and data folders to be output from the file outputter 112. FIG. 9 illustrates a configuration of an anonymized CSV file. FIGS. 8 and 9 are respectively identical to FIGS. 6 and 7 except for the anonymization of some information pieces (highlighted by the dashed lines in FIGS. 8 and 9).

First, when anonymization is to be executed, the file outputter 112 outputs image files having file names containing no identification information on the subjects. Specifically, the file outputter 112 replaces the patient IDs in the file names, defined by the user on the output setting screen 42, of the image files to be output, with other character strings from which the subjects are unidentifiable. For example, the file names of the image files A1, A2, ... originally contain the patient ID “p1234” of the subject A, and the file names of the image files B1, B2, ... originally contain the patient ID “p9876” of the subject B without anonymization, as illustrated in FIG. 6. In contrast, when anonymization is to be executed, the file outputter 112 replaces the patient ID “p1234” in the file names of the image files A1, A2, ... with the number “00001”, and replaces the patient ID “p9876” in the file names of the image files B1, B2, ... with the number “00002”, as illustrated in FIG. 8. The replacement numbers, such as “00001” and “00002”, are assigned to the individual subjects without overlaps. In other words, if the patient IDs are contained in the file names, the file outputter 112 replaces the patient IDs with information pieces, such as serial numbers, unique to the individual subjects but not identifying the subjects. This replacement prevents another user from identifying the subjects from the file names of the output image files. Each replacement number may be a number indicating the current timing and having digits representing “year, month, date, time, minute, and second”. In the case where only the numbers indicating the current timings are insufficient to avoid overlaps among the subjects, the assigned replacement number may be a combination of the number indicating the current timing and a serial number unique to each subject.

Second, when the above-described second option is selected in the case of anonymization, the file outputter 112 creates data subfolders for the individual subjects having folder names each containing no patient ID of the corresponding one of the subjects. Specifically, as illustrated in FIG. 8, the file outputter 112 creates, as the data subfolder for the subject A, a data subfolder having the folder name “Folder_00001” containing no patient ID of the subject A. The file outputter 112 also creates, as the data subfolder for the subject B, a data subfolder having the folder name “Folder_00002” containing no patient ID of the subject B. The numbers, such as “00001” and “00002”, may be identical to or different from the above-mentioned numbers substituting for the patient IDs in the file names of the image files.

Third, when the above-described third option is selected in the case of anonymization, the file outputter 112 creates CSV files for the individual subjects that contain no patient IDs of the corresponding subjects. Specifically, the file outputter 112 originally outputs CSV files containing patient IDs without anonymization, as illustrated in FIG. 7. In contrast, to execute anonymization, the file outputter 112 deletes the patient IDs from the CSV files, as illustrated in FIG. 9. In a non-anonymization case where the CSV files contain the subjects’ names, the file outputter 112 deletes the names from the CSV files for anonymization, although this case is not illustrated. To execute anonymization, the file outputter 112 defines file names (“Test_00001.CSV”, “Test_00002.CSV” ... in the example in FIG. 6) of the CSV files for the individual subjects such that the file names contain no patient IDs. The file outputter 112 thus anonymizes the CSV files to make the subjects unidentifiable from the CSV files.

As described above, when no anonymization is to be executed, the file outputter 112 adds the subjects’ patient IDs to the file names of image files and CSV files to be output, and to the folder names of data folders to be created, as illustrated in FIG. 6. In contrast, when anonymization is to be executed, the file outputter 112 replaces the subjects’ patient IDs in the file names and the folder names with other character strings not identifying the subjects, as illustrated in FIG. 8. While the file outputter 112 adds patient IDs to CSV files to be created in the case of no anonymization as illustrated in FIG. 7, the file outputter 112 adds no patient IDs to the CSV files in the case of anonymization as illustrated in FIG. 9. In other words, the non-anonymized output information contains identification information on the subjects in specific portions, such as file names, folder names, or data content, while the anonymized output information contains no identification information on the subjects in these portions. The non-anonymized output information is applicable to the use requiring identification information on the subjects, while the anonymized output information is applicable to the use requiring personal information protection for the subjects.

Referring back to FIG. 5, the fifth option “Delete patient IDs / doctor IDs from EXIF” on the output setting screen 42 allows the user to select whether to delete the identification information on the subjects and the diagnosticians from the image-associated information 21 that accompanies one or more captured images 20 selected to be output. As illustrated in FIG. 2, the image-associated information 21 may contain a patient or doctor ID, as the identification information on the subjects and the diagnosticians. On the basis of such image-associated information 21 contained in the image files output from the file outputter 112, another user can identify the subject or diagnostician from the image-associated information 21. To avoid this problem, the user can select the fifth option regarding whether to delete the patient or doctor IDs from the image-associated information 21.

Specifically, when the user does not select the fifth option, the file outputter 112 maintains the patient or doctor IDs without deletion that are contained in the image-associated information 21 in the image files to be output. In contrast, when the user selects the fifth option, the file outputter 112 deletes the patient or doctor IDs from the image-associated information 21 in the image files to be output. The file outputter 112 thus anonymizes the image-associated information 21 in response to the user’s selection, to make the subjects and diagnosticians unidentifiable from the image files.

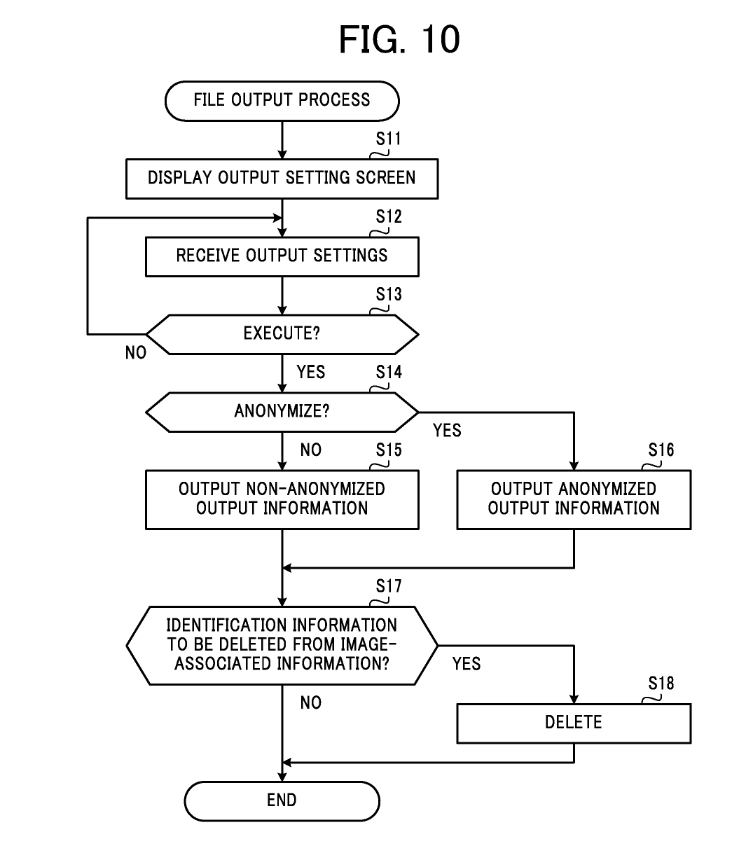

The following describes a file output process executed by the file outputter 112, with reference to FIG. 10. The file output process illustrated in FIG. 10 is initiated when the user selects one or more captured images 20 to be output, from among the captured images 20 appearing in the image field A2 on the viewer screen 40 illustrated in FIG. 3, and then selects the item “Output images”. The file output process illustrated in FIG. 10 is a first example of an information outputting method.

At the beginning of the file output process, the file outputter 112 causes the display 14 to display the output setting screen 42 illustrated in FIG. 5 (Step S11). The file outputter 112 then receives output settings defined by the user on the output setting screen 42 (Step S12). Specifically, the file outputter 112 receives, from the user, the settings of destination folders, output format, and file names of data files to be output, and selection of the options on the output setting screen 42. The file outputter 112 then determines whether the user selects the item “Execute” on the output setting screen 42 (Step S13). When the item “Execute” is not selected (Step S13; NO), the file outputter 112 returns to Step S12 and continues to receive output settings.

In contrast, when the item “Execute” is selected (Step S13; YES), the file outputter 112 determines whether to anonymize the output information (Step S14). Specifically, when the fourth option “Anonymize patient information” is selected on the output setting screen 42, the file outputter 112 determines to anonymize the output information. When determining not to anonymize the output information (Step S14; NO), the file outputter 112 outputs non-anonymized output information (Step S15). Specifically, the file outputter 112 outputs output information containing identification information on the subjects in the file names, folder names, or data content, as illustrated in FIGS. 6 and 7. In contrast, when determining to anonymize the output information (Step S14; YES), the file outputter 112 outputs anonymized output information (Step S16). Specifically, the file outputter 112 outputs output information containing no identification information on the subjects in the file names, folder names, or data content, as illustrated in FIGS. 8 and 9.

The file outputter 112 then determines whether to delete the identification information from the image-associated information 21 (Step S17). Specifically, when the fifth option “Delete patient IDs / doctor IDs from EXIF” is selected on the output setting screen 42, the file outputter 112 determines to delete the identification information from the image-associated information 21. When determining to delete the identification information from the image-associated information 21 (Step S17; YES), the file outputter 112 deletes the patient and doctor IDs corresponding to the identification information from the image-associated information 21 (Step S18). In contrast, when determining not to delete the identification information from the image-associated information 21 (Step S17; NO), the file outputter 112 skips Step S18. These steps complete the file output process illustrated in FIG. 10.

Referring back to FIG. 3, the item “Compare images” on the viewer screen 40 is selected to output multiple captured images 20 such that the captured images 20 are relatively easy to compare with one another. The item “Compare images” is selected when the user wants to observe the captured images 20 in detail, in particular, compare the captured images 20 with one another. The user selects one or more captured images 20 to be output, from among the captured images 20 appearing in the image field A2, by operating the operation unit 13. Then, when the user selects the item “Compare images”, the print outputter 113 executes the second output process. In the second output process, the print outputter 113 outputs, as output information, a print-image data file indicating the captured images 20 of the subjects. The print image indicates an image to be printed by a printer on a print medium, such as paper or films. The print-image data file is an example of file information (output information as a data file) output from the information output device 10.

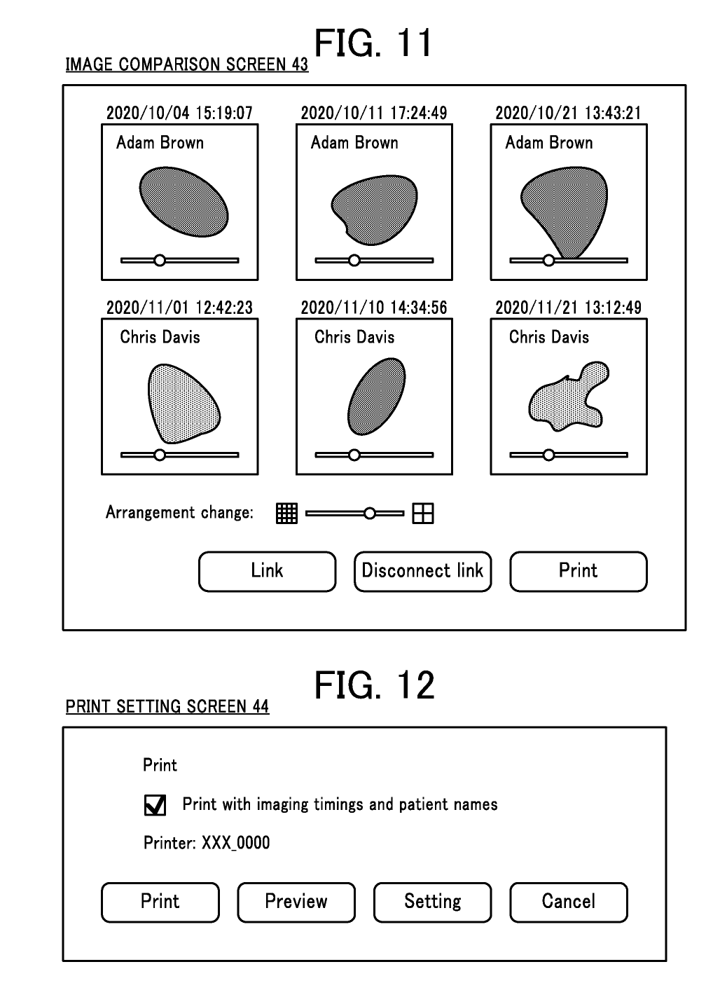

Specifically, when the user selects the item “Compare images” on the viewer screen 40, the print outputter 113 causes the display 14 to display an image comparison screen 43 illustrated in FIG. 11. The image comparison screen 43 allows the user to observe the captured images 20 indicated by the image data pieces 122 stored in the storage 12 while comparing the captured images 20 with one another. The print outputter 113 causes one or more captured images 20, selected to be output on the viewer screen 40, to appear on the image comparison screen 43. In particular, when multiple captured images 20 are selected to be output, the print outputter 113 displays the selected captured images 20 arranged in the column and row directions on the image comparison screen 43, thereby facilitating the user’s observation and comparison. The print outputter 113 shows, for each captured image 20, the imaging timing of the image 20 and the name of the subject captured in the image 20 on the image comparison screen 43. The imaging timings and the subject names related to the captured images 20 can be obtained from the image-associated information 21 accompanying the captured images 20, or the diagnostic record data pieces 123.

The print outputter 113 receives, from the user, a change operation for changing the display mode of each captured image 20 on the image comparison screen 43. Specific examples of the change operation include an operation for zooming in or out each captured image 20, an operation for vertically or horizontally displacing the position of the candidate lesion in each captured image 20, and an operation for changing the arrangement of the captured images 20. The print outputter 113 shows a scroll bar for each captured image 20 on the image comparison screen 43. The scroll bar serves as an interface for changing the display mode of each captured image 20, specifically, zooming in or out or scrolling the captured image 20 independently. The user moves the scroll bar for each captured image 20 using the operation unit 13, thereby zooming in or out the candidate lesion in each captured image 20 or vertically or horizontally displacing the position of the candidate lesion.

The print outputter 113 also shows an arrangement change bar for changing the arrangement of the captured images 20 on the image comparison screen 43. The user moves the arrangement change bar, thereby changing the arrangement of the captured images 20 appearing on the image comparison screen 43. Specifically, in the example in FIG. 11, the captured images 20 are disposed in 3×2 arrangement (such that three images in the row direction and two images in the column direction). The user can change this arrangement of the captured images 20 on the image comparison screen 43 into 2×3, 1×6, or 6×1 arrangement, for example, by moving the arrangement change bar. When the print outputter 113 receives a user’s change operation, such as zooming-in, zooming-out, scrolling, or arrangement-change operation, then the print outputter 113 changes the display mode of the captured image 20 in response to the received change operation. This function helps the user to observe the candidate lesions in the captured images 20.

The print outputter 113 also shows the items “Link”, “Disconnect link”, “Print”, and other items selectable by the user on the image comparison screen 43. The item “Link” is selected to change the display modes of multiple captured images 20 simultaneously. For example, the user selects two or more captured images 20, selects the item “Link”, and then moves the scroll bar for any one of the captured images 20 to input a change operation, such as zooming-in, zooming-out, or scrolling operation. In accordance with the input change operation, the print outputter 113 changes the display mode of this captured image 20. In conjunction (linkage) with this change, the print outputter 113 also changes the display modes of the others of the two or more captured images 20. In other words, when the user inputs a change operation to a single captured image 20, the print outputter 113 zooms in or out the other captured images 20 at the same magnification ratio as that of the captured image 20 that receives the change operation, or scrolls the other captured images 20 by the same distance. Since the display modes of the two or more captured images 20 are changed in conjunction with one another, the user can easily compare the candidate lesions in the two or more captured images 20 with one another at the same magnification ratio, and observe the corresponding positions in detail. By selecting the item “Disconnect link”, the user can turn off the mode of “Link”.

The item “Print” on the image comparison screen 43 is selected to print the captured images 20 appearing on the image comparison screen 43 onto a print medium. As described above, the user can change the display mode of one or more captured images 20 appearing on the image comparison screen 43, by zooming in, zooming out, or scrolling the captured images 20 or changing their arrangement. The user, who wants to print the one or more captured images 20 on the image comparison screen 43 in accordance with the changed display mode, then selects the item “Print”. The one or more captured images 20 can thus be printed on a print medium in accordance with the display mode on the image comparison screen 43.

In more detail, when the user selects the item “Print”, the print outputter 113 causes the display 14 to display a print setting screen 44 illustrated in FIG. 12. The print setting screen 44 allows the user to define print settings for the captured images 20 appearing on the image comparison screen 43. The user can select whether to anonymize the print-image data file, as output information, on the print setting screen 44. Specifically, the user can select whether to anonymize the print image, using the option “Print with imaging timings and patient names” on the print setting screen 44. When the user selects this option and selects the item “Print”, the print outputter 113 generates a print-image data file indicating the captured images 20 selected to be output in accordance with the options selected by the user, and outputs the generated data file to a printer via the communicator 15. The print outputter 113 then transmits a print instruction to the printer, and causes the printer to print the print image on a print medium.

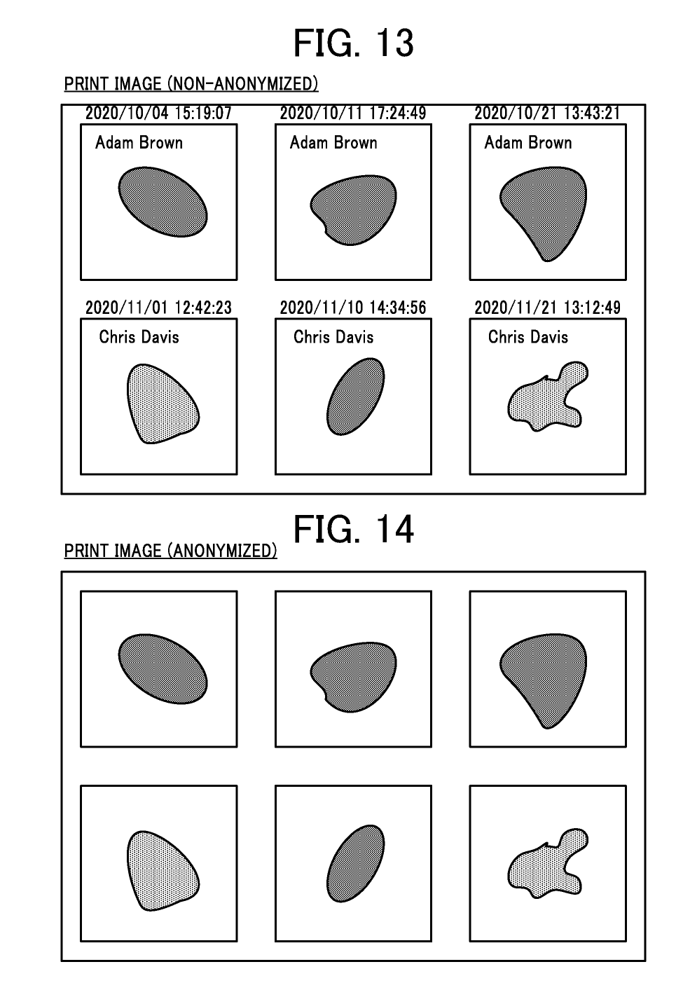

Specifically, FIGS. 13 and 14 each illustrate a print image when the item “Print” is selected on the screen including the 3×2 arrangement of the captured images 20 as illustrated in FIG. 11. When the print image is not to be anonymized, the print outputter 113 generates a print-image data file containing identification information on the subjects as illustrated in FIG. 13, and outputs the generated data file to the printer. The print image illustrated in FIG. 13 contains, for each captured image 20, its imaging timing and the subject name related to the captured image 20, which corresponds to identification information on the subject. The imaging timing and the subject name are identical to the information shown with each captured image 20 on the image comparison screen 43. That is, the print outputter 113 maintains the imaging timings and the subject names shown with the captured images 20 on the image comparison screen 43 to be the same in the print image. The print outputter 113 outputs such a print-image data file containing the imaging timings and the subject names to the printer, and causes the printer to print the data file on a print medium. The resulting print image allows the user to observe and compare the captured images 20 printed on the print medium with one another, while confirming the imaging timings and the subjects related to the captured images 20.

In contrast, when the print image is to be anonymized, the print outputter 113 generates a print-image data file containing no identification information on the subjects as illustrated in FIG. 14, and outputs the generated data file to the printer. Unlike the print image illustrated in FIG. 13, the print image illustrated in FIG. 14 does not contain the imaging timings or the subject names related to the individual captured images 20. In this case, the print outputter 113 deletes the imaging timings and the subject names, shown with the captured images 20 on the image comparison screen 43, from the print image. The print outputter 113 outputs such a print-image data file containing no imaging timings or no subject names to the printer, and causes the printer to print the data file on a print medium. The resulting print image prevents another user from identifying the subjects from the captured images 20 printed on the print medium, thereby protecting personal information for the subjects.

The non-anonymized print image on the print medium is applicable to the use requiring identification information on the subjects, while the anonymized print image is applicable to the use requiring personal information protection for the subjects. The print outputter 113 receives a user’s selection to anonymize or not to anonymize the print image using the option on the print setting screen 44. The user can thus flexibly select whether to anonymize the print image depending on the use. The print outputter 113 may output the print-image data file as a data file in the portable document format (PDF: registered trademark), for example, without outputting the data file to the printer to be printed on a print medium. When the print image is not to be anonymized, the print outputter 113 may add not the subject names but the subjects’ patient IDs in the print image. In contrast, when the print image is to be anonymized, the print outputter 113 does not add the subject names or patient IDs in the print image to make the subjects unidentifiable from the print image.

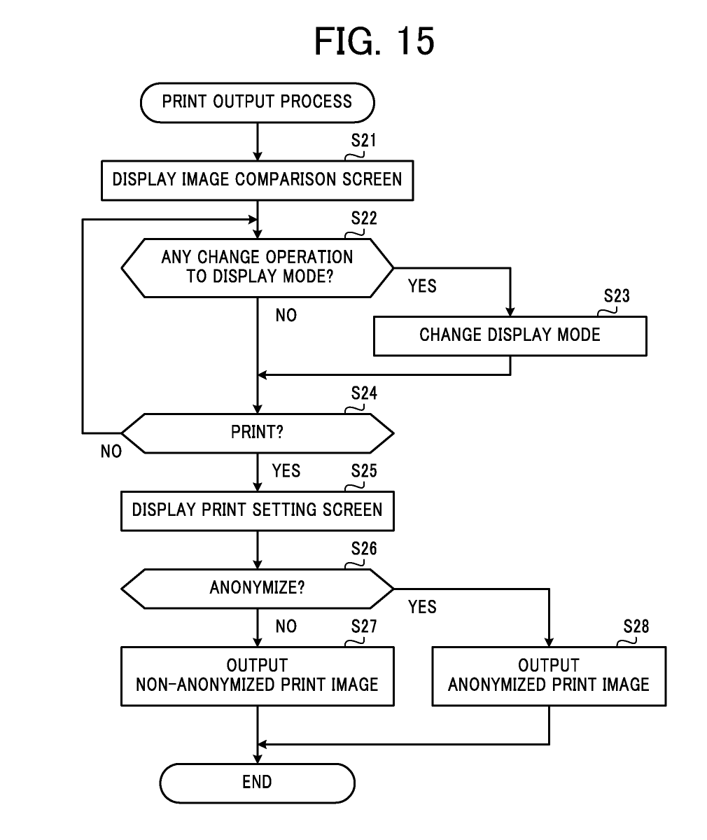

The following describes a print output process executed by the information output device 10, with reference to FIG. 15. The print output process illustrated in FIG. 15 is initiated when the user selects one or more captured images 20 to be output, from among the captured images 20 appearing in the image field A2 on the viewer screen 40 illustrated in FIG. 3, and then selects the item “Compare images”. The print output process illustrated in FIG. 15 is a second example of the information outputting method. At the beginning of the print output process, the print outputter 113 causes the display 14 to display the image comparison screen 43 illustrated in FIG. 11 (Step S21). The print outputter 113 then determines whether the print outputter 113 receives a user’s change operation for changing the display modes of the captured images 20 on the image comparison screen 43, such as zooming-in, zooming-out, scrolling, or arrangement-change operation (Step S22). When receiving the change operation (Step S22; YES), the print outputter 113 changes the display modes of the captured images 20 in accordance with the received change operation (Step S23). In contrast, when receiving no change operation (Step S22; NO), the print outputter 113 skips Step S23.

The print outputter 113 then determines whether the user selects the item “Print” on the image comparison screen 43 (Step S24). When the item “Print” is not selected (Step S24; NO), the print outputter 113 returns to Step S22. The print outputter 113 then continues to receive a change operation, and changes the display modes of the captured images 20 in accordance with the received operation. In contrast, when the item “Print” is selected (Step S24; YES), the print outputter 113 displays the print setting screen 44 illustrated in FIG. 12 (Step S25). The print outputter 113 determines whether to anonymize the print image on the print setting screen 44 (Step S26). Specifically, the print outputter 113 determines to anonymize the print image when the option “Print with imaging timings and patient names” is not selected on the print setting screen 44.

When determining not to anonymize the print image (Step S26; NO), the print outputter 113 outputs a non-anonymized print image (Step S27). Specifically, the print outputter 113 outputs a print-image data file containing the imaging timings and the subject names related to the captured images 20 to the printer as illustrated in FIG. 13, and causes the data file to be printed on a print medium. In contrast, when determining to anonymize the print image (Step S26; YES), the print outputter 113 outputs an anonymized print image (Step S28). Specifically, the print outputter 113 outputs a print-image data file containing no imaging timings or subject names related to the captured images 20 to the printer as illustrated in FIG. 14, and causes the data file to be printed on a print medium. These steps complete the print output process illustrated in FIG. 15.

As described above, before outputting medical diagnostic information, the information output device 10 according to the embodiment receives, from a user, a selection to anonymize or not to anonymize the output information. In response to the selection not to anonymize the output information is received, the information output device 10 outputs the output information without anonymization. In response to the selection to anonymize the output information is received, the information output device 10 outputs anonymized output information. The medical diagnostic information, typically containing sensitive information on the subjects, needs to be anonymized in some cases. The information, however, needs to be accompanied with identification information on the subjects in other cases. In view of these backgrounds, the information output device 10 according to the embodiment receives a user’s selection to anonymize or not to anonymize medical diagnostic information before being output, and thus allows the user to select whether to anonymize output information at the user's discretion. The output information can thus be applicable to both the situations requiring anonymization and the situations not requiring anonymization. The information output device 10 according to the embodiment can accordingly output versatile medical diagnostic information.

The above-described embodiment of the present disclosure is a mere example and not to be construed as limiting the application scope of the present disclosure. That is, the embodiment of the present disclosure may be provided with various modifications, and any modified embodiment can be encompassed in the scope of the present disclosure. For example, although the identification information on a subject is the patient ID or name in the above-described embodiment, the identification information may also be information that identifies the subject other than the patient ID or name. Typical medical diagnostic information in existing techniques contains sensitive information, such as subject’s personal information, and needs to be anonymized to make the subject unidentifiable in some cases. In contrast, some uses require identification information on the subject. The desired objective is to provide versatile medical diagnostic information. The technique of the present disclosure can provide versatile medical diagnostic information. The technique of the present disclosure can output anonymized file information in response to the user’s selection, and thus prevent identification information that may be contained in the file information from being unnecessarily exposed, thereby reducing the possibility that subjects are identified from the file information when the information is provided to the outside. The technique of the present disclosure enables the user to determine not to execute anonymization, which involves some calculations (including conversions and deletions), and can thus conserve the electric power and resources of the CPU and memory dedicated to the anonymization.

Although the captured images 20 capture candidate lesions existing on the subjects’ skins in the above-described embodiment, this configuration is a mere example. The captured images 20 may also capture portions of the subjects other than the skins. For example, the captured images 20 may be images, such as endoscopic images, X-ray images, computed tomography (CT) images, magnetic resonance imaging (MRI) images, or ultrasonic images, capturing candidate lesions existing in portions of the subjects’ bodies other than the skins. Alternatively, the captured images 20 may capture targets other than candidate lesions provided that the captured images 20 are applicable to the diagnosis of the subjects.

Although the information output device 10 includes the functional components illustrated in FIG. 1 in the above-described embodiment, these functional components do not necessarily exist in a single device. For example, the functional components may exist separately in independent devices, such as servers or other devices installed away from each other. In this modification, these devices can be collectively called an information output system. For example, the viewer program 121, and a part or all of the image data pieces 122 and the diagnostic record data pieces 123 may be stored in an external device independent from the information output device 10. In this modification, the processing unit 11 communicates with the external device via the communicator 15 to reference or acquire the data stored in the external device. Alternatively, the display 14 may be included in an external device independent from the information output device 10, and the processing unit 11 may cause the display 14 of the external device to display a screen, such as the viewer screen 40. In these modifications, the system including the external device can be called an information output system.

In the above-described embodiment, the CPU of the processing unit 11 executes the program stored in the ROM or the storage 12, and thus serves as the functional components including the operational processor 111, the file outputter 112, and the print outputter 113. The processing unit 11 may also be dedicated hardware. Examples of the dedicated hardware include single circuits, combined circuits, programmed processors, application specific integrated circuits (ASICs), field-programmable gate arrays (FPGAs), and combinations thereof. In the case where the processing unit 11 is dedicated hardware, the individual functions of the components may be performed by separate pieces of hardware or may be collectively performed by a single piece of hardware. A part of the functions of the components may be achieved by dedicated hardware, whereas the other part may be achieved by software or firmware. That is, the processing unit 11 is able to perform the above-described functions by means of hardware, software, firmware, or a combination thereof.

Programs that define the above-described operation of the information output device 10 may be applied to an existing computer, such as personal computer or cloud server, and may cause this computer to serve as the information output device 10. Such programs may be distributed by any procedure. For example, the programs may be stored in computer-readable recording media, such as compact disk ROMs (CD-ROMs), digital versatile disks (DVDs), magneto optical (MO) disks, or memory cards, and distributed. The programs may also be distributed via a communication network, such as the Internet.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

1. An information processing method, comprising:
receiving, from a user, a selection to anonymize or not to anonymize file information; and
outputting the file information without anonymization in response to the selection not to anonymize the file information is received; or
outputting the anonymized file information in response to the selection to anonymize the file information is received.

2. The information processing method according to claim 1, wherein the anonymization indicates conversion or deletion of a specific information piece in the file information.

3. The information processing method according to claim 1, wherein
the file information to be output is an image file indicating a captured image of a subject, and
the image file has a file name containing
identification information on the subject in response to the selection not to anonymize the file information is received, or
no identification information on the subject in response to the selection to anonymize the file information is received.

4. The information processing method according to claim 1, wherein
data subfolders for individual subjects are created,
the file information is output to a data subfolder for a subject related to the file information among the created data subfolders, and
each of the data subfolders has a folder name containing
identification information on a corresponding subject among the subjects in response to the selection not to anonymize the file information is received, or
no identification information on a corresponding subject among the subjects in response to the selection to anonymize the file information is received.

5. The information processing method according to claim 3, wherein
the image file also indicates image-associated information accompanying the captured image, and
the identification information is deleted from the image-associated information in accordance with a selection received from the user.

6. The information processing method according to claim 1, wherein
the file information to be output is an image data file indicating a captured image of a subject, or a data file indicating diagnostic information on the subject, and
the image data file or the data file contains
identification information on the subject in response to the selection not to anonymize the file information is received, or
no identification information on the subject in response to the selection to anonymize the file information is received.

7. An information processing device to output file information on medical diagnosis, the information processing device comprising:
one or more processors to
receive, from a user, a selection to anonymize or not to anonymize the file information, and
output the file information without anonymization in response to the selection not to anonymize the file information is received, or
output the anonymized file information in response to the selection to anonymize the file information is received.

8. An information processing system to output file information on medical diagnosis, the information processing system comprising:
one or more processors to
receive, from a user, a selection to anonymize or not to anonymize the file information, and
output the file information without anonymization in response to the selection not to anonymize the file information is received, or
output the anonymized file information in response to the selection to anonymize the file information is received.
